(19) United States
(12) Patent Application Publication
(10) Pub. No.: US 2013/0112202 A1
(43) Pub. Date: May 9, 2013

(54) USER INTERFACE FOR BREATHING APPARATUS

(71) Applicant: Petter Fogelbrink, Upplands Vasby (SE)

(72) Inventor: Petter Fogelbrink, Upplands Vasby (SE)

(21) Appl. No.: 13/696,663

(22) PCT Filed: May 7, 2010
(86) PCT No.: PCT/SE2010/050505

§ 371 Date: Jan. 18, 2013

(51) Int. Cl. A61M 16/00

(52) U.S. Cl. CPC A61M 16/0057
USPC 128/204.21

(57) ABSTRACT

A user interface for a breathing apparatuse has a contact-sensitive screen operable to cause a change in the operation of the breathing apparatus based on touch input from an operator. The contact-sensitive screen has a first touch area through which a breathing apparatus setting can be changed by the touch of the operator, and at least a second touch area at a distance from the first touch area. To avoid unintentional changes of the breathing apparatus setting, the user interface is configured such that the breathing apparatus setting can be changed only by touching the first and second touch areas simultaneously.

### BACKGROUND OF THE INVENTION

1. Field of the Invention

The present invention relates to a user interface for a breathing apparatus of the type having a contact-sensitive screen that is operable to cause a change in the operation of the breathing apparatus based on a touch input from an operator, wherein the contact-sensitive screen includes a touch area through which a breathing apparatus setting can be changed by the touch of the operator, and at least one further touch area located on the screen at a distance from the aforementioned touch area.

2. Description of the Prior Art

Contact-sensitive screens, or touch screens, are often used in breathing apparatuses, such as ventilators, in order to provide a user interface through which an operator can interact with the breathing apparatus. When the operator wants to change a breathing apparatus setting related to the operation of the breathing apparatus, the touch screen is normally used only to select the parameter that is to be set, and, sometimes, to confirm the new setting of the parameter. The act of changing the setting, however, is normally performed by rotating a rotary knob, pressing a +/− button, or sliding a slide button, which knob or button is located external to the screen on the breathing apparatus body.

Thus, a typical chain of actions for changing a breathing apparatus setting includes the steps of:
selecting the setting to be changed by touching a touch area associated with the setting on the touch screen, typically realized as a button carrying a name of the setting or the parameter to which the setting relates;
rotating a rotary knob or pressing/sliding a button on the breathing apparatus body to change the setting to a desired value, and
confirming the new setting by touching another touch area on the touch screen, typically realized as a “confirm” or an “accept” button, or by pressing the rotary knob.

A ventilator in which ventilator settings are changed in accordance with the above principle is disclosed in U.S. Pat. No. 5,678,539.

Although not commonly used in the art, there are also user interfaces allowing an operator to change breathing apparatus settings using nothing but a touch screen of the breathing apparatus. This is advantageous in that the entire process can be performed using one and the same device. However, compared to the two-device solution requiring a breathing apparatus operator to use both the touch screen and e.g. a rotary knob of the apparatus in order to change breathing apparatus settings, the risk for unintentional changes of breathing apparatus settings is increased. Unintentional changes of critical breathing apparatus settings may have undesired effects on the on-going respiratory treatment provided to a patient by the breathing apparatus, and even pose a risk to the safety of the patient.

### SUMMARY OF THE INVENTION

It is an object of the invention to provide a Human-Machine Interface (HMI) for a breathing apparatus offering a simple but yet safe way of changing breathing apparatus settings.

This is achieved by a user interface for a breathing apparatus, having a contact-sensitive screen through which a user can change breathing apparatus settings relating to the operation of the breathing apparatus. The contact-sensitive screen has a first touch area through which a breathing apparatus setting can be changed by the touch of a user, and at least a second touch area at a distance from said first touch area. The user interface is configured such that the breathing apparatus setting can only be changed by touching the first and second touch areas simultaneously.

Thus, the invention presents a user interface comprising a contact-sensitive screen, i.e. a touch screen, through which a breathing apparatus setting can be changed. To avoid unintentional changes of the settings and hence unintentional changes in the operation of the breathing apparatus, the user interface is configured such that a two-point contact with the touch screen is required to change the breathing apparatus setting. Thereby, the breathing apparatus setting and the operation of the breathing apparatus can be changed by an operator in a simple and safe way by interacting with nothing but the contact-sensitive screen, i.e. without having to interact with devices such as rotary knobs or buttons which are arranged external to the contact-sensitive screen on the breathing apparatus body.

The first touch area may be a contact-sensitive control element through which the user can change a breathing apparatus setting only if simultaneously touching the second touch area. The second touch area can hence be said to form a “dead man's handle” which has to be touched in order for the operator to change a breathing apparatus setting through the first touch area. Thereby, the first and second touch areas form a two-point touch control through which breathing apparatus settings can be changed. Since the first and second touch areas are separate areas located at a distance from each other on the contact-sensitive screen, the risk of unintentionally changing the breathing apparatus setting becomes small. Preferably, the distance between the first and second touch areas is at least 1 cm, and even more preferably at least 2 cm, to minimize the risk that the operator makes simultaneous contact with both the first and the second touch areas if unintentionally touching the contact-sensitive screen with a finger. That the distance between the first touch area and the second touch area should be at least 1 cm means that there should be at least 1 cm to the closest point of the second touch area from any point of the first touch area.

As used herein, touch area means an area of a contact-sensitive screen within which a touch by a user can be detected by the device of which the screen forms a part.

In some embodiments, the first and second touch areas are located at a distance from each other which is sufficiently short to allow an operator of the breathing apparatus to touch the first and second touch areas simultaneously using only one hand. To this end, the distance from any point of the first touch area to the closest point of the second touch area should preferably be within the range of 1-15 cm, and even more preferably within the range of 1-5 cm. In these embodiments, the first and second touch areas form a two-point touch control intended for one-handed operation.

In other embodiments, the first and second touch areas are located at a longer distance from each other requiring the operator to use two hands in order to change breathing apparatus settings. In these embodiments, the first and second touch areas form a two-point touch control intended for two-handed operation.

In one embodiment of a two-point touch control intended for one-handed operation, the first touch area is realized as a contact-sensitive slide control which is curved and located in relation to the second touch area such that a finger can be moved along the slide control while simultaneously contacting the second touch area with the thumb during a rotary motion of a hand.

In another embodiment of a two-point touch control intended for one-handed operation in which the first touch area is realized as a contact-sensitive slide control, both the slide control and second touch area are substantially straight, elongated areas running in parallel at a short distance from each other, such that the operator can change the breathing apparatus setting by simultaneously sliding two fingers of the same hand up and down the slide control and the second touch area.

According to another embodiment of the invention, the contact-sensitive screen has a number of second touch areas, each associated with a particular breathing apparatus setting, and a single first touch area comprising a contact-sensitive control means, such as a contact-sensitive slide control, contact-sensitive +/− buttons, or a contact-sensitive rotary knob, through which any of the plurality of breathing apparatus settings can be changed by touching the second touch area associated with that particular setting while manipulating the contact-sensitive control means of the first touch area.

According to yet another embodiment of the invention, the contact-sensitive screen comprises a plurality of first touch areas, each associated with a certain breathing apparatus setting, and a single second touch area which, when touched, allows the breathing apparatus setting associated with any of the plurality of first touch areas to be changed. The second touch area may be located close to an edge of the contact-sensitive screen to allow the operator to make continuous contact with the second touch area while resting his hand against a supporting surface external to the screen, such as a frame of a display unit of which the screen forms a part. In this way, the contact-sensitive screen is “unlocked” by touching (and holding) the second touch area, whereupon the various breathing apparatus settings can be changed through the respective first touch areas.

Another object of the invention is to provide a breathing apparatus the operation of which may be changed in a simple but yet safe manner.

This object is achieved by a breathing apparatus having a user interface as described above. The breathing apparatus has a display unit of which the contact-sensitive screen forms a part, and a display control unit for receiving and processing data input by an operator on the contact-sensitive screen, and for causing information to be displayed on the screen in response thereto. The display control unit is configured to determine whether the first and second touch areas are touched or not, and to change the breathing apparatus setting in response to a touch of the first touch area only if the second touch area is touched simultaneously.

Yet another object of the invention is to provide a method offering a simple but yet safe way of changing the operation of a breathing apparatus.

This object is achieved by a method for changing a breathing apparatus setting affecting the operation of a breathing apparatus, including the steps of:
displaying, on a contact-sensitive screen, a first touch area through which said breathing apparatus setting can be changed by the touch of the operator;
displaying, on said contact-sensitive screen, a second touch area at a distance from said first touch area,
determining whether said first and second touch areas are touched or not, and
changing the breathing apparatus setting only in response to simultaneous touches of the first and second touch areas.

### BRIEF DESCRIPTION OF THE DRAWINGS

FIG. 1 illustrates a breathing apparatus according to an exemplary embodiment of the invention.

FIG. 2 illustrates a user interface for a breathing apparatus according to an exemplary embodiment of the invention.

FIG. 3 illustrates a user interface for a breathing apparatus according to another exemplary embodiment of the invention.

FIG. 4 illustrates a user interface for a breathing apparatus according to another exemplary embodiment of the invention.

FIG. 5 illustrates a user interface for a breathing apparatus according to yet another exemplary embodiment of the invention.

### DESCRIPTION OF THE PREFERRED EMBODIMENTS

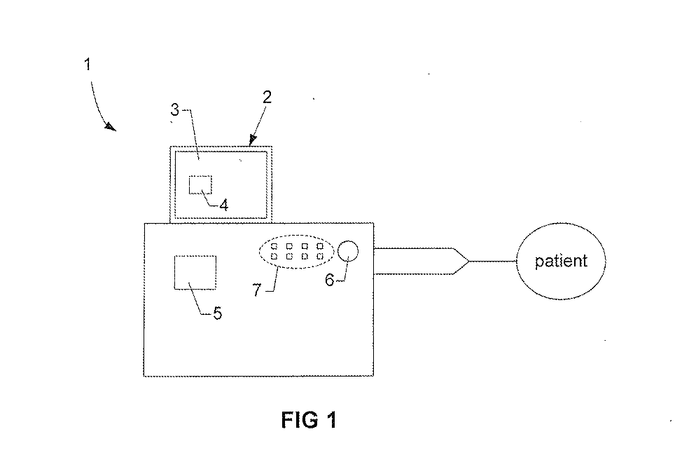

FIG. 1 illustrates a breathing apparatus 1 comprising a display unit 2 having a contact-sensitive screen 3 through which a breathing apparatus operator can change breathing apparatus settings affecting the operation of the breathing apparatus, thereby adjusting the respiratory treatment of a patient connected to the breathing apparatus. The breathing apparatus 1 may be a ventilator, an anesthesia apparatus or any other breathing apparatus configured to provide respiratory care to a patient through the supply of breathing gas.

The contact-sensitive screen 3 is a multi-touch screen providing a user with the ability to apply multiple finger gestures (hereinafter referred to as “touches”) simultaneously onto the screen to send commands to the breathing apparatus 1. The display unit 2 comprises a display control unit 4 for receiving and processing data input by an operator on the contact-sensitive screen 3, and for causing information to be displayed on the screen in response thereto. The display control unit 4 is further operable to cause a change in the operation of the breathing apparatus 1 based on information input by the operator through the screen 3. The display control unit 4 may to this end be connected to a central control unit 5 of the breathing apparatus, which is configured to effectuate the change in operation of the breathing apparatus based on the information input by the operator. For example, the central control unit 5 may be configured to control different valves in the breathing apparatus 1 to alter the flow rate or pressure with which the breathing gas is supplied to the patient, the composition of the breathing gas supplied to the patient, the positive end-expiratory pressure (PEEP) applied to the patient by the breathing apparatus, etc., in response to signals received from the display control unit 4.

In addition to the contact-sensitive screen 3 of the display unit 2, the breathing apparatus 1 may comprise other means through which the operator can change breathing apparatus settings. For example, the breathing apparatus may comprise a rotary knob 6 and/or buttons 7, arranged external to the display unit 3 on the breathing apparatus body.

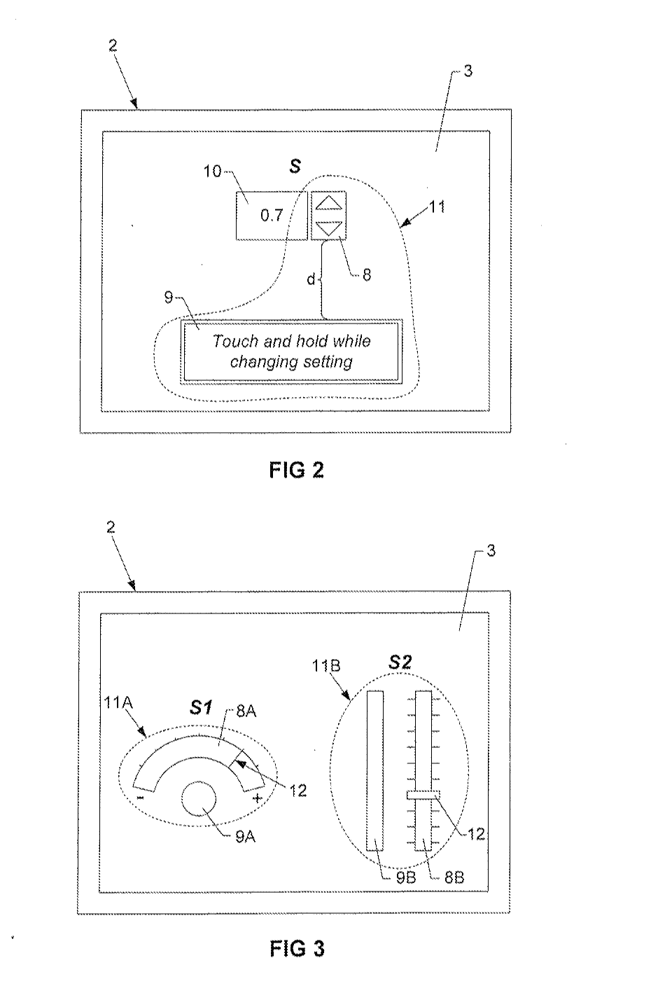

FIG. 2 illustrates an exemplary view of a user interface for the breathing apparatus 1 shown in FIG. 1, as presented on the contact-sensitive screen 3 of the display unit 2. In this view, the screen 3 comprises a first 8 and a second 9 touch area, located at a distance d from each other, through which the operator can change a breathing apparatus setting denoted S. The screen 3 also comprises a current setting indication area 10 in which the current value of the breathing apparatus setting S is displayed. In this exemplary view, the setting S is the setting of a parameter having a current value of 0.7.

The first touch area 8 has two arrows forming contact-sensitive control means through which the setting S can be changed. However, in order to avoid unintentional changes of the setting S, it can only be changed by touching the first touch area 8 while simultaneous contact is made with the second touch area 9, i.e. if the operator touches the first and second touch areas simultaneously. The first 8 and second 9 touch areas can hence be said to form a “two-point touch control” 11 for the setting S, and the second touch area 9 can be said to serve as a “dead man's handle” which has to activated (i.e. touched) in order for the first touch area 8 to function as intended.

The distance d should be long enough to prevent the operator from unintentionally making simultaneous contact with the first 8 and second 9 touch areas. Preferably, the distance d between the two touch areas 8, 9 should be at least 1 cm, corresponding approximately to the width of a fingertip.

With simultaneous reference to FIGS. 1 and 2, the display control unit 3 may be configured to keep track of whether the second touch area 9 is activated (i.e. touched) or not, and to effectuate a change in operation of the breathing apparatus 1 based on a signal received in response to a touch of the first touch area 8 only if the second touch area 9 is activated when receiving the signal.

FIG. 3 illustrates another exemplary view of the user interface for the breathing apparatus 1 shown in FIG. 1, as presented on the contact-sensitive screen 3 of the display unit 2. In this view, two different breathing apparatus settings S1 and S2 can be changed by the operator through a respective two-point touch control 11A, 11B.

The first two-point touch control 11A has a first touch area 8A and a second touch area 9A which, as described above with reference to FIG. 2, have to be touched simultaneously by the operator in order to change the breathing apparatus setting S1. In this embodiment, the first touch area 8A comprises a contact-sensitive control means in form of a contact-sensitive slide control allowing the operator to change the setting S1 by sliding a finger in different directions on the first touch area 8A while simultaneously making contact with the second touch area 9A. The operator can increase the value of S1 by sliding the finger towards a plus sign located at one end of the slide control, and decrease the value of S1 by sliding the finger towards a minus sign at the other end of the slide control. To indicate the current setting to the operator, the first touch area 8A comprises a current setting indicator 12 which is movable along the slide control in response to the slide of a finger on the slide control. The user interface may be realized such that the color of the slide control is different on different sides of the current setting indicator 12, and such that the front between the different colors is moved back and forth over the slide control in response to the sliding movement of the operator's finger. In this way, the operator receives a clear visual indication of the change in the setting S1 resulting from the slide of the finger on the slide control.

The slide control is curved and located in relation to the second touch area 9A such that a finger can be moved along the slide control through a rotary motion of a hand while simultaneously touching the second contact 9A area with the thumb of the same hand. To this end, the elongated slide control is preferably concave in the direction of the second touch area 9A. For example, the slide control may have the shape of a circumferential segment of a circle, and the second touch area 9A may be formed as a button located in or close to the center of said circle.

The second two-point touch control 11B has a first touch area 8B and a second touch area 9B which have to be touched simultaneously by the operator in order to change the breathing apparatus setting S2. In this embodiment, the first touch area 8B comprises a contact-sensitive control means in form of an elongated and substantially straight contact-sensitive slide control. To indicate the current setting to the operator, the slide control comprises a current setting indicator 12 which is movable along the slide control in response to a slide of a finger. The second touch area 9B is formed as an elongated area running alongside said slide control, at a distance therefrom. Thus, the first 9A and second contact 9B areas are formed as two elongated areas which are arranged in parallel in their longitudinal directions. This configuration of the two-point touch control 11B allows the operator to change the setting S2 by sliding two fingers up and down the first 8B and second 9B touch area. Preferably, the distance between the first 8B and second 9B touch areas, in a direction perpendicular to their longitudinal directions, should be short enough to allow the operator to change the setting S2 by means of a sliding movement of a single hand. Even more preferably, the distance should be short enough to allow the operator to change the setting S2 by using two adjacent fingers of a hand, meaning that the distance should be between 1 and 10 cm, and preferably between 1 and 5 cm.

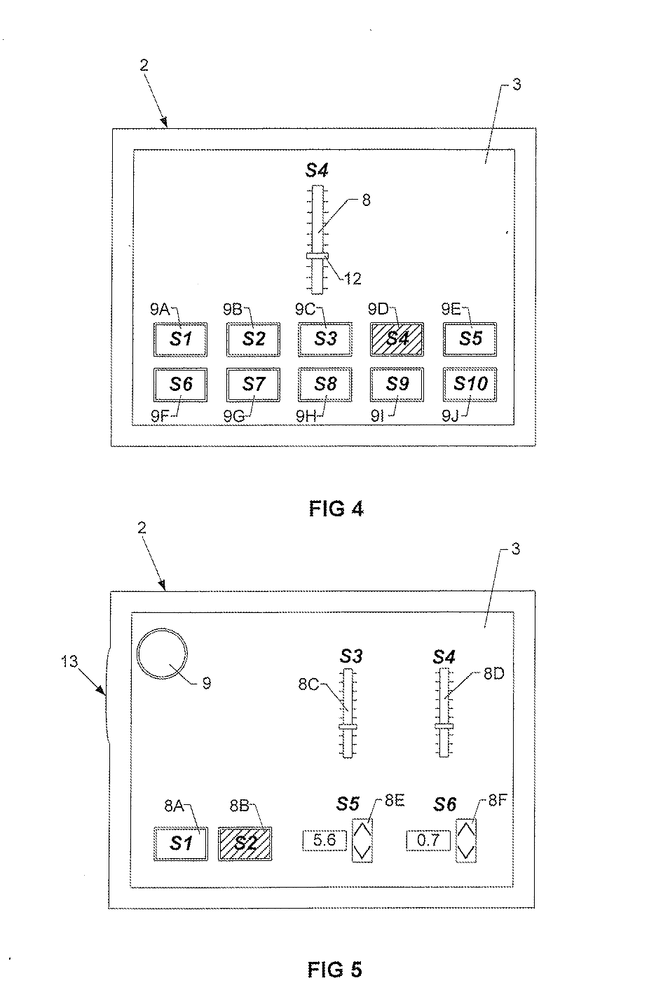

FIG. 4 illustrates another exemplary view of the user interface for the breathing apparatus 1 shown in FIG. 1, as presented on the contact-sensitive screen 3 of the display unit 2. In this view, the contact-sensitive screen 3 has a number (i.e. more than one) of second touch areas 9A-9J, each associated with a certain breathing apparatus setting S1-S10, and a single first touch area 8 allowing the breathing apparatus operator to change the any of the settings S1-S10 by touching the second touch area 9A-9J associated with that setting, and said single first touch area 8, simultaneously. The first touch area 8 has contact-sensitive control means which, in this exemplary case, is realized in form of an elongated and substantially straight slide control as described above with reference to FIG. 3. In this embodiment, the contact-sensitive screen 3 can hence be said to comprise a plurality of two-point touch controls comprising a respective second touch area 9A-9J and a first touch area 8 which is common for all two-point touch controls.

When the operator selects a breathing apparatus setting S1-S10 by touching the second touch area 9A-9J associated with that setting, the visual appearance of the second touch area 9A-9J that is touched may be changed to visually indicate the selection to the operator, e.g. by changing the color thereof. Also, information indicative of the selected breathing apparatus setting may be displayed in or close to the first touch area 8 so as to clearly indicate to the operator which setting may be changed by manipulating the contact-sensitive control means of the first touch area 8 while touching the selected second touch area. Furthermore, when a breathing apparatus setting S1-S10 is selected by the operator by touching one of the second touch areas 9A-9J, the current setting indicator 12 of the slide control may be automatically adjusted to indicate the current value of the selected setting. In the illustrated scenario, the operator has touched, or is touching, the second touch area 9D associated with a breathing apparatus setting denoted S4, the value of which is indicated through the position of the current setting indicator 12.

In the embodiments of the user interface described with reference to FIGS. 2 and 3, the second touch areas 9, 9A-B are “passive” areas in the sense that they serve no purpose other than preventing the operator from unintentionally changing a breathing apparatus setting. In this embodiment, however, the second touch areas 9A-9J serve the double purpose of preventing the operator from unintentionally changing a breathing apparatus setting, and selecting one of a plurality of settings S1-S10, the value of which is to be displayed and/or changed.

Since the distance between the first touch area 8 and some of the second touch areas 9A-9J may be too long in order for the operator to touch the first touch area 8 and the second touch area 9A-9J simultaneously using fingers of the same hand, changing all or some of the settings S1-S10 may require a two-hand operation by the operator.

FIG. 5 illustrates yet another exemplary view of the user interface for the breathing apparatus 1 shown in FIG. 1, as presented on the contact-sensitive screen 3 of the display unit 2. In this view, the contact-sensitive screen 3 has a number of first touch areas 8A-8F, each associated with a certain breathing apparatus setting S1-S6, and a single second touch area 9 allowing the breathing apparatus operator to change any of the breathing apparatus settings by touching the first touch area 8A-8F associated with that setting, and said single second touch area 9, simultaneously. If the second touch area 9 is not touched, none of the breathing apparatus settings S1-S6 can be changed by touching the first touch areas 8A-8J. If, however, the second touch area 9 is touched (and held), any of the breathing apparatus settings S1-S6 can be changed via the first touch areas 8A-8F. Thus, in this embodiment, the touch and hold of the second touch area 9 serves to “unlock” the contact-sensitive screen 3 such that the breathing apparatus settings S1-S6 can be changed.

In this embodiment, the breathing apparatus settings denoted S3-S6 are settings allowing the operator to change different parameter values, such as the PEEP, the oxygen concentration in the breathing gas etc., while the breathing apparatus settings denoted S1-S2 are settings allowing the operator to switch between different modes of ventilation, e.g. between volume-controlled and pressure-controlled ventilation of a patient. According to another exemplary embodiment, the settings denoted S1-S2 are settings allowing the operator to automatically adapt the operation of the breathing apparatus to different patient categories. The first touch areas denoted 8C-8F are respective contact-sensitive control elements in form of a slide-control or set of buttons through which the parameter settings S3-S6 can be changed. The first touch areas denoted 8A-8B, however, is a contact-sensitive control element in the form of a respective button which can be touched by the operator while simultaneously touching the second touch area 9 in order to select a preferred mode of ventilation.

The second touch area 9 is located in the upper left part of the contact-sensitive screen 3 at a location allowing the operator to touch it using his thumb while resting his hand against the frame of the display unit 2. To indicate the intended position of the operator's hand while interacting with the screen 3, the frame of the display unit 2 has a hand position indicator 13 which, for example, may be a convexity formed on the frame of the display unit 2, against which the operator may rest the palm of his left hand while touching the second touch area 9 with the thumb of the same hand. In this embodiment, the user interface is adapted for a right-handed person. However, the contact-sensitive screen 3 may have a corresponding second touch area (not shown) on the right-hand side of the screen 3, instead or in addition to the second touch area 9, so as to adapt the user interface to a left-handed person. According to other embodiments, the user interface may be realized such that it allows the operator to select the position of the second touch area 9 on the screen 3, for example by allowing the second touch area 9 to be “dragged-and-dropped” on the screen 3 by the operator, or by allowing the operator to indicate a position on the screen 3 for the second touch area 9 prior to use of the breathing apparatus 1.

Preferably, the second touch area 9 is displayed on the contact-sensitive screen 3 in a manner that draws the operator's intention to it when not touched. To this end, the second touch area 9 may be displayed having a first visual appearance when not touched, and a second visual appearance that is different from the first visual appearance when touched. In one embodiment, the second touch area may embody a blinking element, such as a circle, which stops blinking when the second touch area is touched by the operator. In another embodiment, the second touch area 9 is realized as a button having a first color that is changed into a second color when touched. In this way, even a breathing apparatus operator using the proposed user interface for the first time may be guided in the direction of touching the second touch area 9.

Similar solutions as the one described above with reference to FIG. 5 can be used in any of the previously described embodiments in order to help the inexperienced operator to interact with the breathing apparatus 1 using the proposed user interface. In any of the previously described embodiments, the second touch area or areas, generally denoted herein by reference numeral 9, may be displayed having a first visual appearance when not touched, and a second visual appearance when touched by the operator. As mentioned above, the first visual appearance should preferably call for the operator's attention, e.g. by including a blinking element or an element in a prominent color, while the second visual appearance should indicate to the operator that touching the second touch area is a positive thing, e.g. by making the blinking element stop blinking or changing the color of the element to a less prominent color. To further increase the usability and intuitive understanding of the proposed user interface, the first touch area or areas, generally denoted herein by reference numeral 8, may also be displayed having a first visual appearance when the second touch area with which the first touch area is associated (i.e. the second touch area with which the first touch area forms a two-point touch control) is not touched, and a second visual appearance when said second touch area is touched by the operator. Here, the first visual appearance should indicate to the operator that some action has to be taken in order to “activate” the first touch area, i.e. in order to render possible change of a breathing apparatus setting through that first touch area. The second visual appearance should indicate that the first touch area is now “active”, i.e. that the breathing apparatus setting can be changed by interaction with the first touch area. To this end, the first visual appearance of the first contact area may include an element, such as a button or a slide-control, which appears semi-transparent and/or in a first, less prominent color on the contact-sensitive screen 3, whereas the second visual appearance may include the same element appearing opaque and/or in a second, more prominent color on the screen 3.

In the above description of exemplary views of the user interface according to the invention, the steps involving interpretation and processing of data input by the operator via the contact-sensitive screen 3 are typically performed by the display unit control unit 4 of the breathing apparatus, shown in FIG. 1. Likewise, the steps involving display of information on the contact-sensitive screen based on data input by the operator, or data relating to the operation and/or status of the breathing apparatus, are typically also performed by the display control unit 4. As described with reference to FIG. 1, the breathing apparatus may also have a central control unit 5 with which the display control unit 4 may be configured to cooperate to control the operation of the breathing apparatus 1 based on data input by the operator via the contact-sensitive screen 3, and to display information relating to the operation of the breathing apparatus 1 on the contact-sensitive screen.

It should also be appreciated that the first touch area may be any form of contact-sensitive control element through which the breathing apparatus setting can be changed while simultaneously touching the associated second touch area, and that the control means is not limited to contact-sensitive buttons and slide controls as described with reference to the exemplary embodiments of the invention. For example, the contact-sensitive control elements may also be realized in form of a switch that can be placed in any of two or more discrete positions by a finger gesture applied onto the first touch area, or in form of a (digital) rotary knob which may be “rotated” by the operator by a finger gesture applied onto the first touch area.

Although modifications and changes may be suggested by those skilled in the art, it is the intention of the inventor to embody within the patent warranted heron all changes and modifications as reasonably and properly come within the scope of his contribution to the art.

I claim as my invention:

1-16. (canceled)

17. A user interface for a breathing apparatus, comprising:
a contact-sensitive screen configured to respond to touch inputs made by an operator;
a processor in communication with said contact-sensitive screen and configured to be in communication with a breathing apparatus to emit signals to said breathing apparatus dependent on entries made by touch inputs through said contact-sensitive screen;
said contact-sensitive screen comprising a first touch area configured to generate a setting for said breathing apparatus in response to a touch of said first touch area by an operator, and at least one second touch area located at said contact-sensitive screen at a distance from said first touch area; and
said processor being configured to emit said setting to said breathing apparatus only in response to a simultaneous touch by an operator to said first touch area and said second touch area.

18. A user interface as claimed in claim 17 wherein said first touch area comprises a contact-sensitive control element selected from the group consisting of a contact-sensitive button, a contact-sensitive slide control, a contact-sensitive switch, and a contact-sensitive rotary knob.

19. A user interface as claimed in claim 17 wherein said first and second touch areas are located at said distance from each other allowing an operator to simultaneously touch said first and second touch areas using only one hand.

20. A user interface as claimed in claim 19 wherein said first touch area comprises a contact-sensitive slide control that is curved and is located relative to said second touch area to allow a finger of the operator to be moved along said slide control while simultaneously contacting said second touch area with a thumb by implementing a rotary motion of a hand of the operator.

21. A user interface as claimed in claim 19 wherein said first touch area comprises a contact-sensitive slide control, said slide control and said second touch area being substantially straight, elongated areas proceeding in parallel at said distance from each other, said distance allowing an operator to change said setting by sliding two fingers of a same hand along said slide control and said second touch area.

22. A user interface as claimed in claim 17 wherein said contact-sensitive screen comprises a plurality of second touch areas, each associated with a respective breathing apparatus setting, and wherein said first touch area is a common touch area for all of said second touch areas and comprises a contact-sensitive control element with which any of the plurality of respective breathing apparatus settings can be changed by simultaneously touching a respective one of said second touch areas associated with a breathing apparatus setting that is to be changed, and said contact-sensitive control element of said first touch area.

23. A user interface as claimed in claim 17 wherein said contact-sensitive screen comprises a plurality of first touch areas, each associated with a respective breathing apparatus setting, and a common second touch area for said plurality of first touch areas, each of said plurality of first touch areas comprising a contact-sensitive control element with which the associated breathing apparatus setting can be changed by simultaneously touching the respective contact-sensitive control element and the common second touch area.

24. A user interface as claimed in claim 23 wherein said common second touch area is located at an edge of said contact-sensitive allowing an operator to make continuous contact with said second touch area while resting a hand against a supporting surface external to said contact-sensitive screen.

25. A user interface as claimed in claim 17 wherein at least one of said first touch area and said second touch area comprises an element having a first visual appearance when said second touch area is not touched, and having a second visual appearance, different from said first visual appearance, when said second touch area is touched.

26. A user interface as claimed in claim 17 wherein said breathing apparatus setting is a critical setting relating to an ongoing respiratory treatment of a patient provided by said breathing apparatus.

27. A user interface as claimed in claim 26 wherein said breathing apparatus setting is selected from the group consisting of a determinant for a composition of a breathing gas delivered to the patient by the breathing apparatus, a target pressure affecting operation of said breathing apparatus, a target flow affecting operation of said breathing apparatus, and a mode of ventilation in which said breathing apparatus operates.

28. A breathing apparatus comprising:
a breathing-assist device configured to interact with a patient to provide breathing assistance and/or anesthesia to the patient;
a contact-sensitive screen configured to respond to touch inputs made by an operator;
a processor in communication with said contact-sensitive screen and with said breathing-assist device to emit signals to said breathing-assist device dependent on entries made by touch inputs through said contact-sensitive screen;
said contact-sensitive screen comprising as first touch area configured to generate a setting for said breathing-assist device in response to a touch of said first touch area by an operator, and at least one second touch area located at said contact-sensitive screen at a distance from said first touch area; and
said processor being configured to emit said setting to said breathing-assist device only in response to a simultaneous touch by an operator to said first touch area and said second touch area.

29. A method for operating a breathing apparatus, comprising:
providing a contact-sensitive screen configured to respond to touch inputs made by an operator;
via a processor in communication with said contact-sensitive screen emitting signals to said breathing apparatus in communication with said processor dependent on entries made by touch inputs through said contact-sensitive screen;
providing said contact-sensitive screen with a first touch area configured to generate a setting for said breathing apparatus in response to a touch of said first touch area by an operator, and at least one second touch area located at said contact-sensitive screen at a distance from said first touch area; and
from said processor, emitting said setting to said breathing apparatus only in response to a simultaneous touch by an operator to said first touch area and said second touch area.

30. A method as claimed in claim 29 comprising:
displaying said second touch area at said screen with a first visual appearance when said second touch area is not touched; and
displaying said second touch area at said screen with a second visual appearance, different from said first visual appearance, when said second touch area is touched.

31. A method as claimed in claim 29 comprising
displaying said first touch area at said screen with a first visual appearance when said second touch area is not touched; and
displaying said first touch area at said screen with a second visual appearance that is different from said first visual appearance, when said second touch area is touched.
